(19) United States
(12) Patent Application Publication
(10) Pub. No.: US 2019/0082117 A1
(43) Pub. Date: Mar. 14, 2019

(54) INTRAVASCULAR ULTRASOUND IMAGE PROCESSING OF BLOOD-FILLED OR BLOOD-DISPLACED LUMENS

(71) Applicant: ACIST Medical Systems, Inc., Eden Prairie, MN (US)

(72) Inventors: Thomas C. Moore, Livermore, CA (US); Kendall R. Waters, Sammamish, WA (US)

(21) Appl. No.: 15/704,710

(22) Filed: Sep. 14, 2017

(51) Int. Cl. H04N 5/243; G06T 7/00; H04N 5/235; G06T 5/20; G06T 5/00; H04N 5/232; A61B 8/12; A61B 8/08

(52) U.S. Cl. CPC H04N 5/243; G06T 2207/20008; H04N 5/2351; G06T 5/20; G06T 5/002; H04N 5/23219; A61B 8/12; A61B 8/0891; A61B 8/5207; A61B 8/5269; G06T 2200/28; G06T 2207/10152; G06T 2207/30024; G06T 2207/30101; G06T 2207/10132; G06T 7/0014

(57) ABSTRACT

Techniques for intravascular ultrasound image processing of blood-filled or blood-displaced lumens are disclosed. A catheter assembly may include an intravascular imaging device with an imaging element to image a vasculature and generate imaging data. An imaging engine, including a programmable processor, may communicate with the intravascular imaging device. The imaging engine may determine a lumen state of the vasculature, the determined lumen state indicative of whether the vasculature is blood-filled or blood-cleared. The imaging engine may perform signal processing to enhance the generated image data. Finally, the imaging engine may generate an image based on the enhanced imaging data and the determined lumen state.

### TECHNICAL FIELD

This disclosure is related to the field of intravascular imaging and processing of intravascular image data.

### BACKGROUND

Intravascular imaging is often used to identify diagnostically significant characteristics of a vessel. For example, an intravascular imaging system may be used by a healthcare professional to help identify and locate blockages or lesions in a vessel. Common intravascular imaging systems include intravascular ultrasound (IVUS) systems as well as optical coherence tomography (OCT) systems.

IVUS systems include one or more ultrasound transducers emitting ultrasound energy based on received electrical signals and sending return electrical signals based on ultrasound energy reflected by various intravascular structures. In some instances, a console with a high-resolution display is able to display IVUS images in real-time. In this way, IVUS can be used to provide in-vivo visualization of vascular structures and lumens, including the coronary artery lumen, coronary artery wall morphology, and devices, such as stents, at or near the surface of the coronary artery wall. IVUS imaging may be used to visualize diseased vessels, including vessels with coronary artery disease. In some instances, the ultrasound transducer(s) can operate at a relatively high frequency (e.g., 10 MHz-60 MHz, in an embodiment, 40 MHz-60 MHz) and can be carried near a distal end of an IVUS catheter assembly. Some IVUS systems involve 360-degree visualization of the vessel (e.g., mechanically rotating the IVUS catheter assembly, steering IVUS signals from phased-array transducers, etc.).

Electrical signals received by the transducer can represent image information and can be used to construct images. In some systems, analog image information can be digitized into vector form. An image can then be constructed from a series of vectors. For example, M vectors each comprising N data points can be used to construct an M×N two-dimensional image. In some systems, images of vascular structures of a patient can be generated and displayed in real-time to provide in-vivo visualization of such structures.

An ultrasound transducer typically produces analog signals and operates at a particular frequency. Generally, the resolution of the image information received increases with the operating frequency of the transducer and the frequency of data acquisition by the transducer; that is, high frequency images tend to have better resolution than low frequency images. However, data acquired at a high frequency often includes greater signal loss, and thus a lower signal to noise ratio (“SNR”) when compared to low-frequency images because of losses associated with high-frequency transmission. This can result in dark, hard-to-see images or very noisy images if the image intensity is amplified via increased gain. As a result, most intravascular imaging is performed at a relatively low frequency, sacrificing image resolution for an improved SNR.

In some systems, image information is processed to improve the SNR. Processing can include combining data such as averaging, envelope detection, and/or selecting various data points to eliminate, such as outliers. However, each processing step takes time. For example, in some systems, envelope detection can require each vector to be passed through the envelope detector one-by-one, slowing down the imaging process. If the processing delay is too long, it can become impossible to generate a real-time display for in-vivo visualization of the vascular structures being imaged.

### SUMMARY

Embodiments include an intravascular imaging system (e.g., IVUS) that automatically detects whether blood has been displaced from an imaged vessel and adjusts image signal processing accordingly. If the signal provided by the imaging transducer indicates that blood was present in the imaged vessel when imaged, the steps in processing the signal into an image can each possess a corresponding set of attributes. If, on the other hand, the signal provided by the imaging transducer indicates that blood was displaced from the imaged vessel when imaged, one or more steps in processing the signal into an image can possess a different set of attributes. The differences in the image processing steps can account for differences in how imaging energy (e.g., ultrasound) propagates through blood vs. through the fluid used to displace the blood. Examples of blood displacement fluid include saline, contrast, Ringer's solution, dextran, and lactate solution.

As noted, in some embodiments, the imaging system also adjusts the signal processing, based on whether blood has been displaced, to generate a more accurate image of the vessel. The intravascular imaging system performs high frequency image acquisition and effective noise filtering of the entire range of noise. Processing steps are performed to achieve high resolution, low noise images. A sufficiently low degree of noise permits the image information to be amplified to show high-resolution detail without also amplifying the noise to a point at which the image becomes obscured.

In some embodiments, such processing steps can include time gain compensation, coherence filtering of high frequency data, envelope detection to convert the high frequency data to low frequency data, envelope vector averaging, spatial filtering of low frequency data, gamma correction, and frame filtering. Some processes, such as envelope detection, can be performed in parallel to expedite processing. In some embodiments, processing steps are performed quickly enough to generate and display a high-resolution image from high frequency image information in real time.

Systems for performing such measurements can include an intravascular imaging catheter assembly configured to generate a raw frame of imaging information corresponding to its surroundings during data collection, for example, a patient's vasculature. The raw frame of imaging information can include a raw set of vectors, each vector in the raw set of vectors including a raw set of data points. In some instances, each vector is representative of an angular portion of image information, while each data point within the vector is representative of a radial dimension along that angular portion. The brightness of each vector in the raw set of vectors may be monitored; when a sufficient quantity of pixels become dark, the system can infer that the blood has been cleared from the imaged vessel. The system can include an imaging engine for receiving the raw frame of imaging information from the intravascular imaging catheter assembly and producing an enhanced frame of imaging information that includes an enhanced set of vectors.

The imaging engine may perform near-field artifact reduction. The imaging engine can include a coherence filter configured to group vectors from the raw set of vectors into raw vector groups and to generate a first set of vectors based on comparisons of data points within the raw vector groups. In some cases, the comparisons are between points of like radial position within each vector. Vectors in the first set of vectors are each generally representative of the vectors in one of the raw vector groups and include a first set of data points. The first set of data points within each vector in the first set of vectors can include the same number of data points as each set of raw data points in the raw imaging information.

The imaging engine can include an envelope detection module for receiving the first set of vectors and generating a second set of vectors based on comparisons of data points within each first set of data points with one another. Each vector in the second set of vectors can include a second set of data points. Each second set of data points can have a smaller number of data points than its associated first set, but can be representative of the first set of data points. In some cases, the second set of data points can include a lower-frequency representation of the first set of data points. The second set of vectors can include the same number of vectors as the first set.

The imaging engine can include a spatial filter for receiving the second set of vectors and generating an enhanced set of vectors. The spatial filter can group vectors from the second set of vectors into processed vector groups, and generate an enhanced set of vectors based on comparisons of data points of each processed vector group. The spatial filter can include comparisons of data points within each processed vector group having like and near radial position. In some examples, each processed vector group can be used to generate a single enhanced vector in the set of vectors. Each enhanced vector can include the same number of data points as the second set of data points in associated vectors in the second set of vectors. The enhanced set of vectors can be combined to produce the enhanced frame of imaging information.

In some systems, the imaging engine can include an image generator configured to generate an image based on the enhanced frame of imaging information. Such systems can include a display coupled to the imaging engine for displaying images generated by the image generator. In some systems, images can be displayed to a user in substantially real time from the image generator and display.

Related technology is disclosed in the following documents: (A) U.S. Pat. No. 9,693,754, filed on May 15, 2013 and entitled “IMAGING PROCESSING SYSTEMS AND METHODS”; (B) U.S. Pat. No. 9,704,240, filed on Oct. 7, 2014 and entitled “SIGNAL PROCESSING FOR INTRAVASCULAR IMAGING”; and (C) U.S. Patent Application Publication No. 2017/0103498, filed on Sep. 30, 2016 and entitled “SYSTEMS AND METHODS TO REDUCE NEAR-FIELD ARTIFACTS.” The entire contents of these documents are incorporated herein by reference.

### BRIEF DESCRIPTION OF THE DRAWINGS

FIG. 1 illustrates an intravascular imaging system, according to an example embodiment.

FIG. 2A illustrates a front view of propagating ultrasound data vectors of a catheter, according to an example embodiment.

FIG. 2B illustrates a cross-sectional view of a catheter within a vessel and an overlay of ultrasound data vectors propagated by the catheter, according to an example embodiment.

FIGS. 3A and 3B illustrate coherence filter profiles as part of the intravascular imaging engine, according to an example embodiment.

FIG. 4 illustrates an envelope detection process, according to an example embodiment.

FIG. 5A illustrates a set of brightness data arranged for display, according to an example embodiment.

FIG. 5B illustrates a subset of image information data, according to an example embodiment.

FIG. 6 is a process flow diagram illustrating a multi-step process to generate high-resolution intravascular images, according to an example embodiment.

FIG. 7 is a data flow diagram illustrating the flow of image information from the transducer to a display, according to an example embodiment.

FIG. 8 is a block diagram illustrating an example of a machine, upon which any one or more example embodiments may be implemented.

### DETAILED DESCRIPTION

The following detailed description is exemplary in nature and is not intended to limit the scope, applicability, or configuration of the invention in any way. Rather, the following description provides some practical illustrations for implementing examples of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

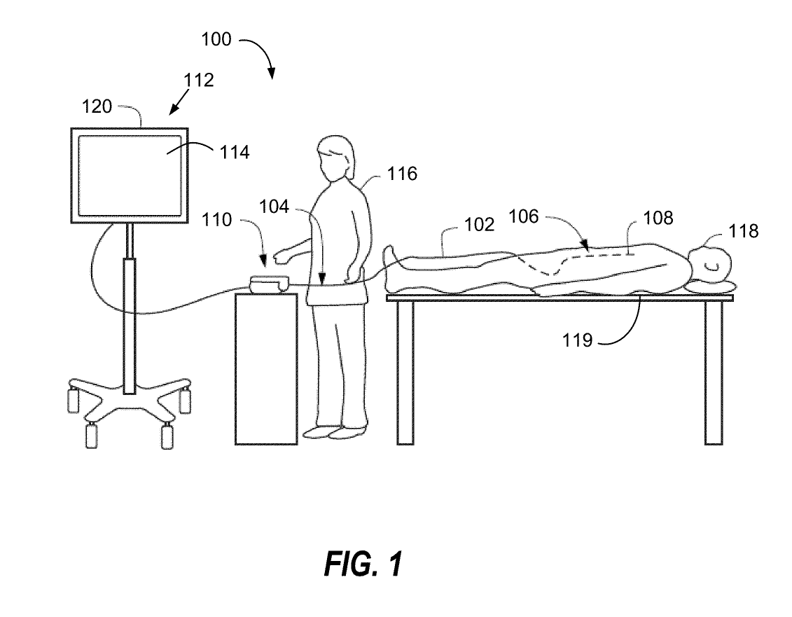

FIG. 1 illustrates an intravascular imaging system 100, according to an example embodiment. System 100 may include a catheter assembly 102, a translation mechanism 110, and a user interface 120. The catheter assembly 102 may include a proximal end 104 and a distal end 106 configured to be inserted into a vessel of a patient 118. In one example, catheter assembly 102 may be inserted into the patient 118 via the femoral artery and guided to an area of interest within the patient 118. The broken lines in FIG. 1 represent portions of catheter assembly 102 within the patient 118.

In some examples, catheter assembly 102 may include a transducer 108 within distal end 106 configured to emit and receive wave-based energy and generate imaging data—e.g., to image the area of interest within the patient 118. For example, where system 100 is an IVUS system, transducer 108 may comprise an IVUS imaging probe including an ultrasound transducer configured to emit and receive ultrasound energy and generate ultrasound data. In another example, system 100 may be an OCT system wherein the transducer 108 may comprise an OCT imaging probe configured to emit and receive light and generate OCT data.

In some embodiments, the catheter assembly 102 can include an imaging assembly and a sheath. The imaging assembly can include the transducer 108, a drive cable, and a transmission line (e.g., a coaxial cable). The sheath can define a lumen within which the imaging assembly is allowed to move freely. The drive cable can be fixed to the transducer 108 such that movement of the drive cable through the sheath causes the transducer 108 to move through the sheath as well. Thus, in some embodiments, the transducer 108 can both translate and rotate within the sheath via the drive cable without the sheath moving within the artery. This can be advantageous to avoid excess friction between the catheter assembly 102 and the interior of a patient's artery as the transducer 108 is moved during imaging or other intravascular imaging operations. For example, while moving inside the sheath, the catheter assembly 102 does not drag along vessels that may have plaques prone to rupture.

The intravascular imaging system 100 can include a translation mechanism 110. As shown, the translation mechanism 110 can be mechanically engaged with the catheter assembly 102 and configured to translate the catheter assembly 102 a controlled distance within the patient 118 during a pullback or other translation operation. In some embodiments, the translation mechanism 110 can act as an interface with the catheter assembly 102. The translation mechanism 110 can translate all or part of the catheter assembly 102 through the vasculature of the patient 118. For example, in an embodiment in which the catheter assembly 102 comprises a drive cable attached to the transducer 108 housed within a sheath, the translation mechanism 110 can act to translate the drive cable and transducer 108 through the sheath while keeping the sheath fixed within a vessel of the patient 118.

The intravascular imaging system 100 can include an intravascular imaging engine 112. In some embodiments, the intravascular imaging engine 112 can include a processor such as a digital signal processor (DSP), an application specific integrated circuit (ASIC), or a field-programmable gate array (FPGA), a user interface 120, memory, a display 114, and so on. The intravascular imaging engine 112 can receive image information from the catheter assembly 102, and in some embodiments, the processor of the intravascular imaging engine 112 can process the image information and/or generate a display based on the image information received from the catheter assembly 102. In various embodiments, the intravascular imaging engine 112 can present the generated display on display 114 and/or store the generated display in memory. In some embodiments, the display 114 can be updated in real-time (or near real-time) to provide in-vivo visualization of the vasculature of the patient 118.

In some embodiments, the user interface 120 can receive commands by a system user 116 and/or display intravascular imaging data acquired from the catheter assembly 102 (e.g., intravascular images). The user interface 120 may include a traditional PC or PC interface with software configured to communicate with the other components of the intravascular imaging system 100. In some embodiments, the user interface 120 may include the display 114, which may be configured to display system information and/or imaging signals from the catheter assembly 102 (e.g., intravascular images). In some embodiments, the user interface 120 includes a touchscreen display, which can act to both receive commands from a system user 116 and display intravascular imaging data from the catheter assembly 102.

Although the intravascular imaging engine 112 can comprise a processor, user interface 120, memory, and a display 114, the intravascular imaging engine 112 can alternatively comprise any combination of these or other components suitable for performing the functions of the intravascular imaging engine 112 disclosed herein. For example, the intravascular imaging engine 112 can comprise a processor configured to receive image information from the catheter assembly 102 and generate a display. In such embodiments, the intravascular imaging engine 112 can be in communication with any of a user interface, a display 114 on which to present the generated display, and/or memory in which to store the generated display if any such component is not part of the intravascular imaging engine 112.

In some embodiments, analog image information from the transducer 108 can be digitized into a series of vectors to be digitally processed. In an exemplary embodiment, a single vector can include N data points, each respective data point corresponding to a respective distance from the transducer 108. Images can be constructed out of M vectors, each vector corresponding to an orientation of a rotatable transducer 108 (e.g., mechanically rotated, phased array, etc.). On a high level, the M vectors of N data points can be used to construct an image with M×N data points in polar coordinates. In some embodiments, each vector comprises information representing an angular section extending outward from the transducer 108. Because of the angular width of the wave-based energy emitted by the transducer 108, it is common for a portion of the imaged angular section of vasculature from one vector to be included in one or more additional vectors. In other words, imaged angular sections represented by different vectors can overlap one another in the course of generating M vectors.

In some embodiments, to construct a vector, the imaging engine can sample data from the transducer 108 at a series of points in time (e.g., N points) and populate the vector with each subsequently received data point. Accordingly, the frequency of data collection corresponds to the vector size, N. As discussed elsewhere herein, a higher frequency image generally has higher resolution but with lower signal levels due to more signal loss, or equivalently, a lower SNR when compared to a lower frequency image. For example, the transmission line of the catheter assembly 102 can act as an antenna and pick up electrical noise from various sources within the environment in which the intravascular imaging system 100 is operating.

In an embodiment, the intravascular imaging engine 112 can be configured to process image information acquired at a high frequency to effectively improve the SNR. In some embodiments, the intravascular imaging engine 112 receives a set of high frequency image information from the transducer 108, comprising M vectors, with each vector comprising N data points. In some embodiments, the high frequency image information is a raw frame of imaging information including a raw set of vectors, each vector of the raw set of vectors including a raw set of data points. For instance, in various examples, high frequency image information can include a raw set of 4096, 2048, or 1024 vectors. Each vector can include a raw set of, for example, 2560 data points. In general, each vector can include any number of data points depending on the imaging system. As discussed, high frequency data often includes a large amount of noise, including high frequency and low frequency noise. In some embodiments, the intravascular imaging engine 112 can perform one or more processing functions to effectively reduce the high and/or low frequency noise from the set of image information.

For example, the intravascular imaging engine 112 can perform one or more calculations for reducing noise in the set of image information. In various examples, one or more calculations can include a comparison of two or more data points within the image information. In general, comparisons of data can include any calculation operation that incorporates a value of the one or more data points being compared. Accordingly, comparisons of data points can include combining values associated with the data points, such as summing, averaging, or determining other data set parameters, such as determining a median value, mode, a minimum value, a maximum value, and the like. Comparisons can further include performing mathematical or other functions involving such data, such as grouping or eliminating of data based on compared values, for example.

In some embodiments, the intravascular imaging engine 112 is configured to receive each vector from the raw set of vectors and perform coherence filtering in order to filter out high frequency noise and improve the SNR of the image information. In some examples, the coherence filter is configured to group vectors from the raw set of vectors into raw vector groups of one or more vectors and to generate a first set of vectors based on comparisons of data in each vector in the raw vector groups. In some examples, the first set of vectors is generated based on comparisons of data points of each vector within the raw vector group with one another at like radial positions. That is, vectors can be compared with one another at like vector coordinates during coherence filtering. In some examples, the comparison can include taking an average of the vectors in the raw vector group at like vector coordinates. In various embodiments, the average can be a weighted average or a standard mean calculation. As a result of the coherence filtering, in some embodiments, each vector in the first set of vectors is representative of vectors of one of the raw vector groups and includes a first set of data points having the same number of data points as the raw sets of data points in each vector in the set of raw vectors.

In an exemplary embodiment, each raw vector group consists of two vectors, each having N data points. In such embodiments, the raw set of vectors can include twice as many vectors as the first set of vectors. Accordingly, raw sets of vectors having 4096, 2048, or 1024 vectors can be filtered into first sets of vectors having 2048, 1024, or 512 vectors, respectively.

In general, coherence filtering can include combining one or more vectors in one or more combinations, for example, averaging. In some embodiments, a set of X vectors are averaged to create a single, average vector. Averaging can be performed, for example, point-wise among averaged vectors. For instance, in embodiments in which each vector corresponds to an angular coordinate in polar coordinates while each vector entry corresponds to the a different radial position in polar coordinates, comparison of two vectors can be performed at each common radial position (e.g., the nth vector entry of one vector is compared with the nth vector entry in another vector).

In some such embodiments, if the transducer 108 provides M total vectors to the intravascular imaging engine 112, the resultant number of vectors after averaging would be M/X. In more complicated embodiments, various forms of weighted averaging or averaging in multiple combinations can be used. In one specific example, a series of four vectors (v1, v2, v3, and v4) can be processed such that four resulting “super vectors” (s1, s2, s3, and s4) are created. One such processing example is as follows:

s1=(Σ(v2,v3,v4))/3

s2=(Σ(v1,v3,v4))/3

s3=(Σ(v1,v2,v4))/3

s4=(Σ(v1,v2,v3))/3

In this example, each possible combination of three unique vectors is used to create a resulting “super vector.” In some embodiments, each sum can be scaled to provide a more traditional average.

In the preceding example, four vectors are processed into four new vectors, each effectively comprising an average of three of the original four. The image information that would be present in all four original vectors is preserved in the new vectors, but the noise that would likely be present in less than all four original vectors would be diminished significantly in the new vectors. Accordingly, each of the resulting vectors has reduced high frequency noise, and the resultant number of vectors after averaging is still M. In an embodiment, the vectors that are averaged represent image information from overlapping or near overlapping sections of the patient's vasculature. In general, any number of vectors of overlapping sections can be combined to produce resultant “super vectors.”

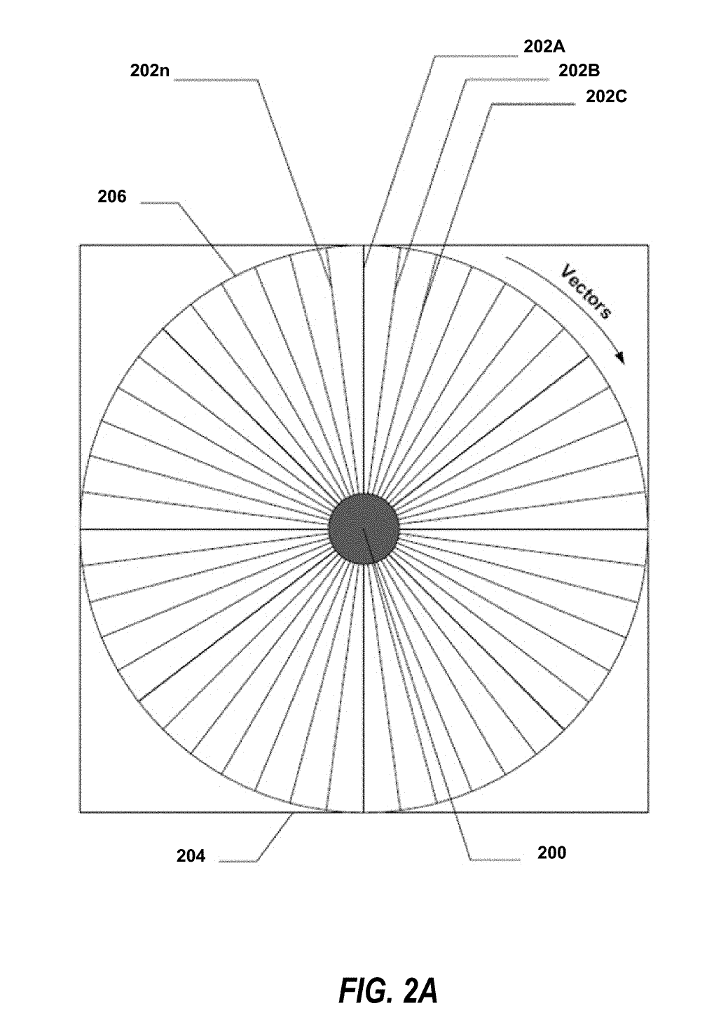

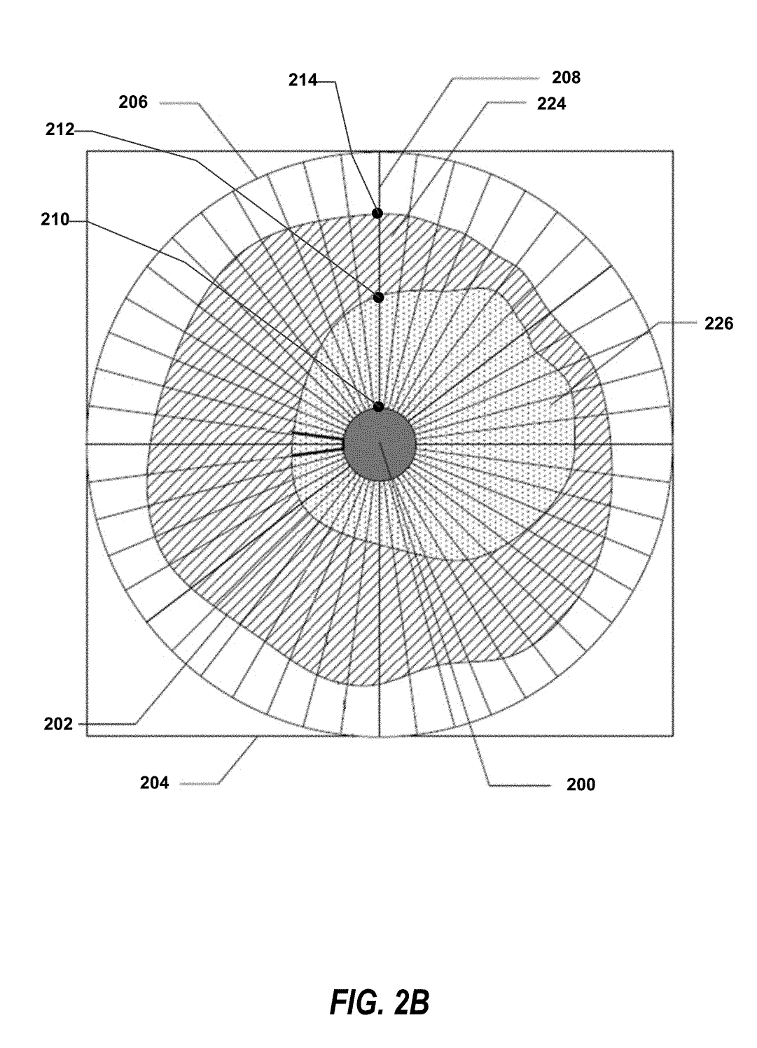

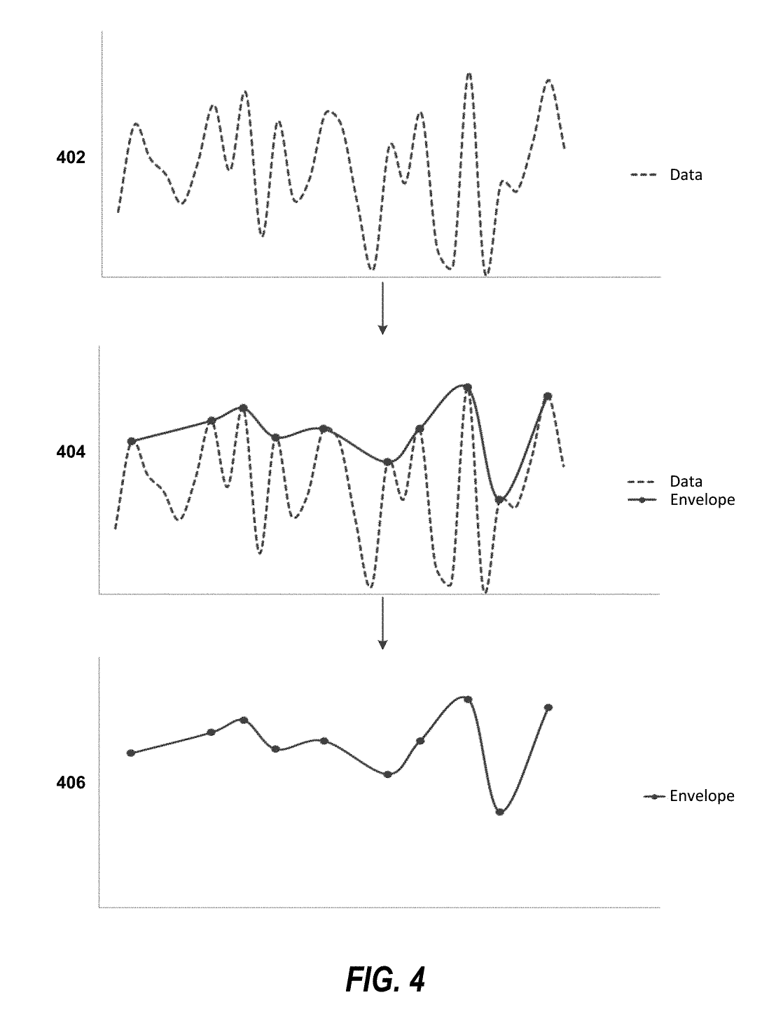

FIG. 2A illustrates a front view of propagating ultrasound data vectors of a catheter 200, according to an example embodiment. In this example, the catheter 200 may be a mechanically rotating ultrasound imaging catheter similar to catheters previously described (e.g., catheter assembly 102). Likewise, the catheter 200 may be configured to rotate an ultrasound transducer (not shown) relative to a sheath of the catheter 200, and the ultrasound transducer may be configured to generate ultrasound data by emitting and receiving acoustic energy. The ultrasound data vectors illustrated in FIG. 4 are indicative of acoustic energy emitted and received by the ultrasound transducer at different rotational positions. More specifically, each data vector is representative of ultrasound data collected by the ultrasound transducer at different rotational positions of the ultrasound transducer. In some embodiments, each of the data vectors can be acquired at different times.

As shown in FIG. 2A, the ultrasound transducer of catheter 200 may generate ultrasound data on a vector-by-vector basis as the transducer is rotated. For example, the ultrasound transducer may initially acquire an ultrasound data vector 202A and continue to acquire vectors 202B through 202n as the ultrasound transducer is rotated clockwise. Accordingly, vectors 202A-202n can be representative of a full 360-degree rotation of the ultrasound transducer within a vessel and make up a single frame. The number of data vectors acquired per rotation may vary depending on the application of the catheter 200. For instance, in some embodiments, the catheter is configured to generate between about 500 and about 5,000 vectors per rotation. For example, in an embodiment generating 512 vectors per rotation (e.g., frame) the angle between data vectors may then be characterized as approximately 2π/512 radians or 360/512 degrees. In an example of a catheter configured to generate 2096 vectors per rotation (e.g., frame), the angle between data vectors may be approximately 2π/2096 radians or 360/2096 degrees. FIG. 2A also provides a representation of a data frame 204 that comprises emitted and received vectors 202A-202n. An imaging view 206 of the catheter 200 may be based on the magnitude of the data vectors propagated by the catheter and may vary to suit a specific application. The magnitude of the data vectors may be based on a number of factors, for example, the frequency of the emitted wave (e.g., 60 MHz) and/or the power level of the wave. In some embodiments, the ultrasound transducer of catheter 200 can emit acoustic energy at differing frequencies within the single data frame 204.

FIG. 2B illustrates a cross-sectional view of a catheter 200 within a vessel 202 and an overlay of ultrasound data vectors propagated by the catheter 200, according to an example embodiment. Vessel 202 may be a vasculature of a patient and catheter 200 may be catheter assembly 102. As in those examples, the catheter 200 may include an ultrasound transducer configured to generate ultrasound data in the form of a plurality of data vectors. In this example, each data vector corresponds to ultrasound data collected by emitting acoustic energy and receiving a reflection of the energy, or backscatter, from vessel 202 and/or items of or within vessel 202. Different portions of the vessel, for example vessel wall 224 as well as fluid (e.g., blood or blood-displacement fluid) and plaque in vessel lumen 226, are likely to have different material compositions. The different material compositions of the different portions of the vessel can result in different responses to the emitted acoustic energy. The different responses of the various portions can be exploited in many embodiments to distinguish different portions, or regions of interest, of the vessel and in turn provide a more diagnostically valuable image.

For instance, variations in ultrasound backscatter levels along a data vector may be used to determine the boundary between the vessel lumen 226 and the vessel wall 224. For example, vessel wall 224 and the fluid within vessel lumen 226 (e.g., blood or blood-displacement fluid) may reflect varying amounts of acoustic energy emitted by the ultrasound transducer of catheter 200. Accordingly, the ultrasound data collected along a data vector may capture the variation in the ultrasound backscatter level between the vessel wall 224 and the vessel lumen 226. For example, a first region of data vector 208 between data points 210 and 212 may have a backscatter level consistent with blood flowing within the vessel lumen 226 while a second region of data vector 208 between data points 212 and 214 may have a backscatter level consistent with vessel wall 224. Further, the transition between the backscatter levels of the first region and the second region may be used to identify the boundary between the vessel wall 224 and the vessel lumen 226, located approximately at data point 212. As noted above, data frame 204 may comprise data vectors acquired during a full 360-degree rotation of the ultrasound transducer of catheter 200. As such, data frame 204 can include imaging data at a cross-section of the vessel 202 within an imaging view 206 that is defined by the particular imaging parameters used in a specific application.

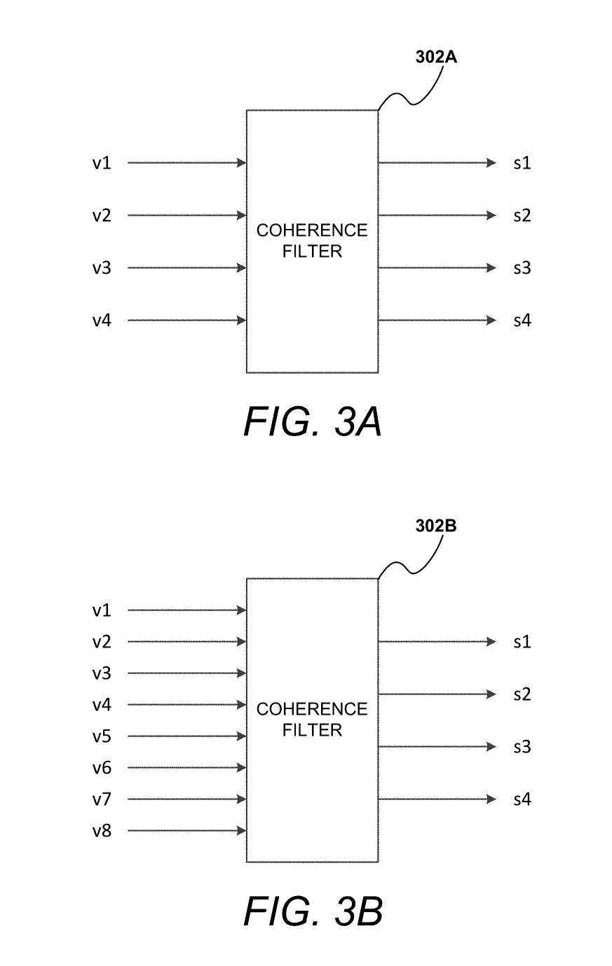

FIGS. 3A and 3B illustrate coherence filter profiles as part of the intravascular imaging engine, according to an example embodiment. FIG. 3A illustrates a coherence filter 302A similar to the one described above receiving four vector inputs (v1, v2, v3, and v4) and outputting four “super vectors” (s1, s2, s3, and s4). In some embodiments, the number of resultant “super vectors” is lower than the number of input vectors, such as shown in FIG. 3B. FIG. 3B illustrates a coherence filter 302B receiving eight input vectors (v1-v8) and outputting only four “super vectors” (s5-s8). In such embodiments, input vectors can be combined in any way to reduce the total number of resultant “super vectors,” such as selecting four possible combinations of two or more input vectors each and performing an averaging function. Many implementations are possible and are within the scope of the coherence filter described in this disclosure. The coherence filter can be configured to output “super vectors” having lower high frequency noise and a higher SNR than the high frequency input vectors produced by the high frequency image information received from the transducer 108.

Referring again to FIG. 1, in some embodiments, vectors can be processed by the intravascular imaging engine 112 using an envelope detection module comprising one or more envelope detectors. The envelope detection module can be configured to receive the first set of vectors from the coherence filter and generate a second set of vectors based on comparisons of data points within each first set of data points with one another. That is, in some examples, the envelope detection module generates a second set of vectors based on comparisons of data points within each vector of the first set of vectors. The envelope detection module can act on each of the first set of vectors independently. Accordingly, in some embodiments, the second set of vectors comprises the same number of vectors as the first set of vectors. The envelope detection module can include a plurality of envelope detectors arranged in parallel for parallel processing of vectors in the first set of vectors. The envelope detectors can be arranged in parallel such that each envelope detector is configured to generate a subset of the second set of vectors.

In some example embodiments, an envelope detector in the envelope detection module can effectively convert vectors comprising high frequency data into vectors comprising low frequency data, while maintaining the general shape of the waveform represented by the vector. FIG. 4 illustrates an envelope detection process, according to an example embodiment. FIG. 4 illustrates a set of high frequency data in frame 402 to be input into the envelope detector. Peaks in the data are detected to create an (upper) envelope of the data in frame 404. Other envelope functions are also possible. The envelope is output as the signal shown in frame 406, having the same general wave shape as the input data with a lower frequency. In some examples, “super vectors” created from the received image information and having reduced high frequency noise can be directed to one or more envelope detectors. In some embodiments, a plurality of envelope detectors can be used in parallel. For example, in an embodiment in which X vectors are processed to create Y “super vectors,” the system 100 can include Y envelope detectors in parallel to process each of the “super vectors” simultaneously. In the exemplary case presented above, four envelope detectors can be used in parallel to process “super vectors” s1-s4 or s5-s8 simultaneously. In such an example, processing time for performing envelope detection is reduced by a factor of four when compared to systems using a single envelope detector.

In embodiments in which the system is configured to display an image representative of the received image information, high frequency vectors can comprise too much data for effective display. The envelope detectors can generally output vectors comprising low frequency brightness data representative of the received information even when the input “super vectors” are high frequency vectors. In this way, envelope detectors can be used to smooth the data and make it suitable for display in the form of low frequency vectors of brightness data.

The low frequency brightness data can be in the form of a series of vectors, each vector comprising a series of data points. The vectors can make up the second set of vectors and the series of data points in each vector can include the second set of data points in each of the vectors. In some embodiments, each vector generally corresponds to an orientation angle of the transducer within the patient, while the data within each vector generally corresponds to information of the patient's vascular structure encompassed within the angular range of the vector at increasing radial distances from the transducer. As noted, because of the angular width of the wave-based energy emitted by the transducer, imaged angular sections represented by differing vectors can overlap one another.

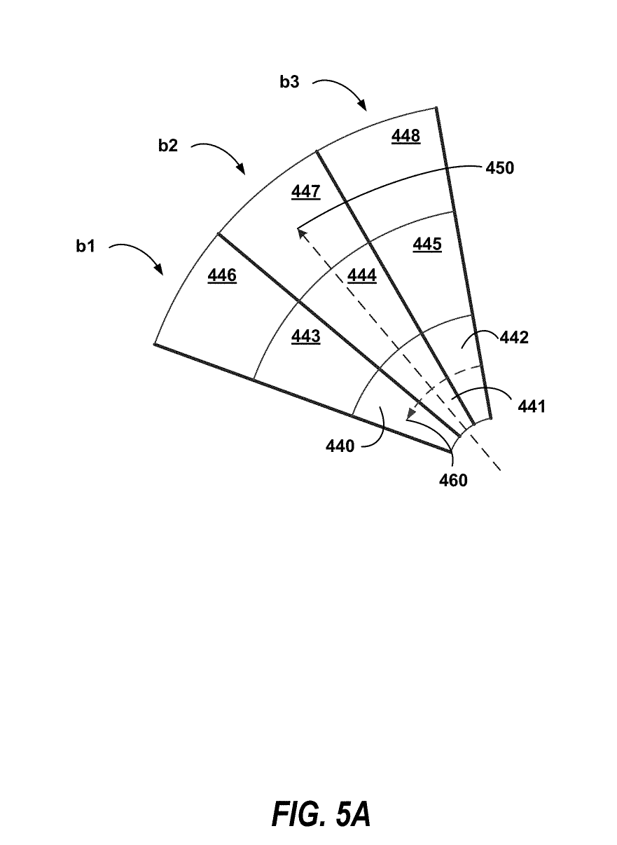

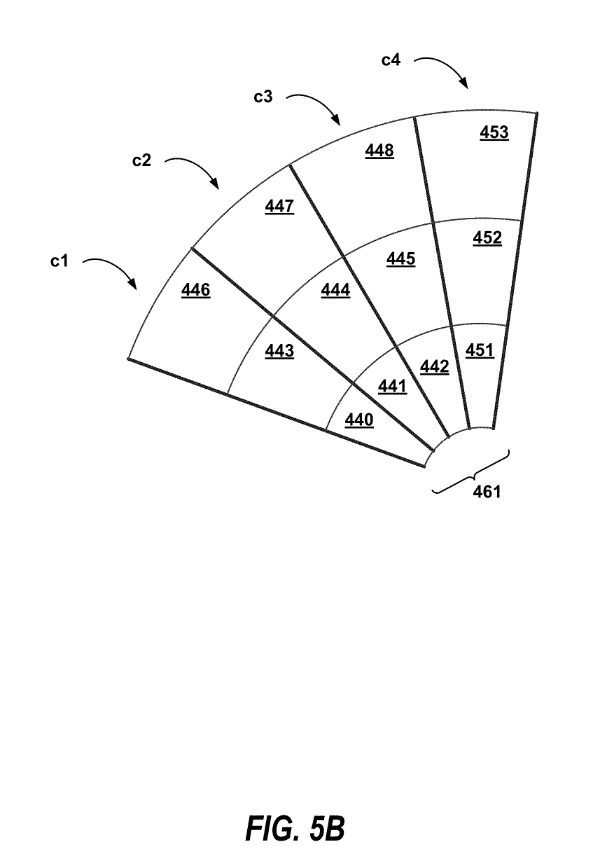

FIG. 5A illustrates a set of brightness data arranged for display, according to an example embodiment. In this example, brightness data is arranged in polar coordinates, with data points 440-448 divided both angularly and radially. Each angular section separated by bold lines represents a vector of brightness data, corresponding to an orientation of the transducer 108 during image information acquisition. Each point within an angular section represents a data point within that vector. For example, in the embodiment shown, brightness vector b1 comprises data points 440, 443 and 446; brightness vector b2 comprises data points 441, 444, and 447; and brightness vector b3 comprises data points 442, 445, and 448.

As discussed, vectors can represent a range of angles to generally comprise data representative of an angular section of a patient's vascular structure. Accordingly, nearby vectors can comprise data representing overlapping sections of vascular structure. A sufficient number of vectors can effectively represent a full 360-degree image of the vascular structure. The number sufficient depends on the angular width subtended by each vector and the amount of overlap of each vector. Vectors and the data points they comprise can, for instance, make up a polar coordinate representation of the imaged vascular structure, with the vector in which a data point is contained corresponding to the angle coordinate of that point, and the location of the data point within the vector corresponding to the radial position of that point.

Vectors comprising low frequency brightness data can be used to generate a display representative of the image information received by the catheter assembly. The image can be displayed in color, black and white, grayscale, or any other desired color palette, and can comprise a set of pixels, each pixel representing a data point of brightness data. The brightness and/or color of each pixel can directly correspond to the brightness data represented in the corresponding data point. In some embodiments, even if high frequency noise has been reduced from the image information such as described with regard to coherence filtering, the low frequency brightness data used to generate the display can contain low frequency noise.

In some embodiments, the intravascular imaging engine can be configured to process and combine the low frequency brightness data in order to reduce the low frequency noise. Such processing can include one or both of filtering and averaging. In some examples, the system can include a spatial filter configured to receive data from the envelope detection module for further processing. The spatial filter can be configured to group vectors of the second set of vectors into processed vector groups. Each of the processed vector groups can include any number of vectors from the second set of vectors.

The spatial filter can be configured to generate an enhanced set of vectors based on data in the second set of vectors. For instance, in some examples, the spatial filter can perform comparisons of data points of each processed vector group's vectors. The comparisons can be performed, for example, at like and near radial positions among the vectors in each processed vector group. Each vector in the resulting enhanced set of vectors can be representative of the vectors in one of the processed vector groups. Each vector in the enhanced set of vectors can include an enhanced set of data points. In some examples, the enhanced set of data points in each vector in the enhanced set of vectors can have as many data points as the second set of data in each vector of the second set of vectors.

In some systems, processing the second set of vectors via the spatial filter can include spatial filtering of brightness data. Examples of spatial filtering can include averaging data points with a set of spatially proximate additional data points. Spatially proximate data points can be data points whose polar coordinate representation are within some predetermined distance of one another. In a 360° image representation, averaged points can include, for example, all data points within a certain spatial distance of a certain point—a technique called proximal averaging. With reference to FIG. 5A, for example, point 444 can be processed to be averaged with each neighboring points 440-448. In various embodiments, the spatial requirement used to define the averaging process can be predetermined or set by a user.

Another example of spatial filtering can comprise averaging all data points within a certain spatial distance of a certain point and within the same vector as the certain point—a technique called radial averaging. For example, points 440, 441, and 442 can be averaged along line 460 to generate spatially filtered data at 441. Yet another spatial filtering example can comprise averaging all data points within a certain distance of a certain point and having the same radial position within their corresponding vector—a technique called angular averaging. For example, points 441, 444, and 447 can be averaged along line 450 to generate spatially filtered data at 444. Spatial filtering can involve a median filter, which can be useful for minimizing the impact of outliers.

In general, spatial filtering can include averaging or other methods of combining data chosen via any other selection of proximate data points. Spatial filtering can include removing outliers from a set of data prior to averaging the remaining set of data. In some embodiments, filtering operations can be determined on a point-by-point basis. For example, not all data points will necessarily have the same number of surrounding data points within a given spatial dimension. Spatially filtering the low frequency brightness data can act to reduce the low frequency noise contained therein, effectively raising the SNR.

Still another example of spatial filtering can be described with regard to FIG. 5B. FIG. 5B illustrates a subset of image information data, according to an example embodiment. In FIG. 5B, four vectors c1, c2, c3, and c4 each include three data points. Each vector (c1-c4) corresponds to an angular coordinate while each data point within each vector corresponds to a radial position in a polar coordinate representation. In some examples in accordance with FIG. 5B, vectors c1-c4 are vectors in the second set of vectors resulting from an envelope detection step. In an exemplary spatial filtering step, vectors c1-c4 can be grouped into a processed vector group 461 by the spatial filter. A processed vector group can in general include any appropriate number of vectors from the second set of vectors, and generally includes a plurality of vectors. In the illustrated embodiment of FIG. 5B, the processed vector group 461 comprises four vectors (c1-c4). The spatial filter can generate an enhanced set of vectors based on comparisons of data points in the vectors within each processed vector group, such as c1-c4.

In an exemplary process, the spatial filter can generate the enhanced set of vectors based on comparisons of data points of each processed vector group's vectors with one another at like and near radial positions (e.g., radial positions including points 440, 443, and 446 in FIGS. 5A and 5B). In some instances, each vector in the enhanced set of vectors is representative of the vectors of one of the processed vector groups. With regard to FIG. 5B, processed vector group 461 can be used to generate a vector in the enhanced set of vectors. In an exemplary method, points at a first radial position (e.g., points 443, 444, 445, and 452) are analyzed, and the data point with the highest value is excluded from the analysis. Then points with neighboring radial positions (e.g., points 440, 441, 442, 451, 446, 447, 448, and 453) are similarly analyzed, and the highest value data point at each radial position is excluded. In accordance with the illustrated embodiment, of the 12 data points (440-448 and 451-453), three are excluded for being the highest value data point at each radial distance. The remaining nine data points can be compared to generate a data point entry for the first radial position in the resulting enhanced vector. For example, the median value of the remaining nine data points can be used as the corresponding data point at the first radial position in the resulting enhanced vector. A similar analysis can be performed at each radial position of the vectors in the second set of vectors, and accordingly, each vector in the enhanced set of vectors can include an enhanced set of data points having the same number of data points as the second set of data points (e.g., the number of data points in each vector in the second set of vectors).

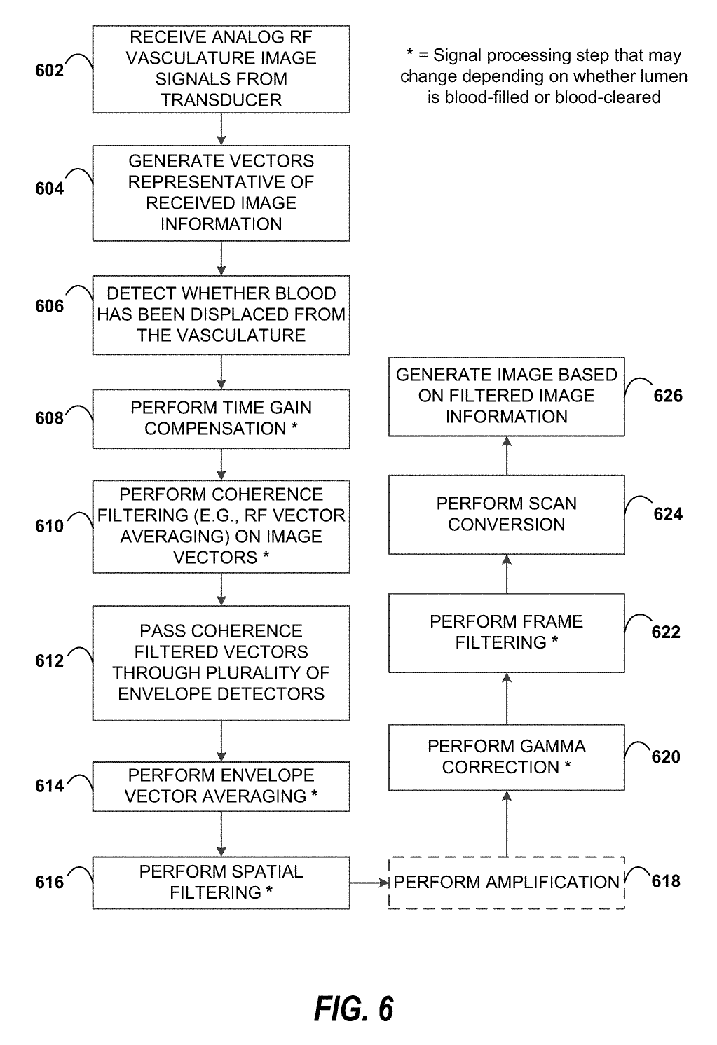

Various processes have been described in which high frequency noise is reduced, high frequency data is converted to low frequency data, and low frequency noise is reduced. In some embodiments, these steps can be aggregated into a multi-step process in order to produce a high-resolution intravascular image. FIG. 6 is a process flow diagram illustrating a multi-step process to generate high-resolution intravascular images, according to an example embodiment. In an exemplary embodiment, an intravascular imaging engine can receive high frequency image information from the transducer (operation 602). In some embodiments, image information is in the form of a series of vectors. In other embodiments, the intravascular imaging engine can generate vectors representative of the received image information (operation 604). Vector generation can comprise digitizing received analog image information from the transducer. In some embodiments, the high frequency image information can comprise raw radio frequency (RF) image information.

The intravascular imaging engine may use low-frequency imaging to detect whether blood has been displaced from the vasculature (operation 606). In some embodiments, this detection is by observing how many pixels near the center of an intravascular image have become dark (e.g., black); when the number (or proportion) of pixels that have become dark cross a threshold, the intravascular imaging engine determines that blood has been displaced from the vasculature. Upon determining that blood has been displaced from the vasculature, the intravascular imaging engine may automatically initiate high-frequency imaging and various operations (e.g., a pullback). Some of the operations may be performed differently, depending upon whether blood has been displaced from the vasculature.

The intravascular imaging engine may perform time gain compensation on the high-frequency image information (operation 608). As a signal travels away from the ultrasound transducer, the signal is attenuated. A signal always becomes attenuated when the signal travels through tissue, and usually becomes attenuated when the signal travels through blood. Therefore, image processing performed using signals through tissue or blood must account for the signal attenuation to generate accurate images. However, a signal undergoes insignificant attenuation when the signal travels through a portion of a vessel whose blood has been displaced; thus, the intravascular imaging engine does not need to perform time gain compensation for a signal that travels within a blood-displaced portion of a vessel.

Speed of Sound Compensation

Ultrasound waves travel through blood at a rate that is slower than through blood displacement fluid (e.g., contrast, saline, etc.). IVUS images are generated based on how the ultrasound waves travel through blood. If the ultrasound waves instead traveled through blood displacement fluid, the resulting image will include some level of inaccuracy because the resulting image is based on a faulty assumption. The algorithm that accounts for the speed of sound in blood may be adjusted to instead account for the speed of sound in the blood displacement fluid (e.g., saline), which results in a more accurate image.

Near-Field Artifact Reduction

The imaging engine may perform near-field artifact reduction, as described in International Patent Application No. US2016/054589, filed on Sep. 30, 2016 and entitled, “SYSTEMS AND METHODS TO REDUCE NEAR-FIELD ARTIFACTS.” When blood is displaced from a vessel, the near-field artifact effect becomes more prominent.

The intravascular imaging engine can process the received high frequency image information using coherence filtering (e.g., a form of averaging) to reduce the high frequency noise (operation 610). The coherence filtering process can produce a series of high frequency “super vectors” representing the image information received from the transducer having reduced high frequency noise.

The “super vectors” can be passed through a plurality of envelope detectors arranged in parallel and configured to simultaneously process an equal plurality of “super vectors” (operation 612). The envelope detectors can convert the high frequency “super vectors” into low frequency brightness vectors comprising brightness data representative of the image information received from the transducer. The brightness vectors can represent image information, for example, in polar coordinates, with each data point of each vector representing an angular and radial component.

The intravascular imaging engine may perform envelope vector averaging on the output data from the envelope detectors (operation 614). Envelope detection is described in greater detail in U.S. Pat. Nos. 9,693,754 and 9,704,240, both of which are incorporated herein by reference above. The imaging engine may then generate an enhanced data vector from the average of the detected envelopes.

Brightness data from the enhanced data vector can be subject to spatial filtering, including, for example, proximal, angular, or radial averaging, in order to reduce the low frequency noise in the low frequency brightness data (operation 616). The intravascular imaging engine can include an image generator configured to generate an image representing the image information received from the transducer 626. The image generator can be configured to generate an image based on an enhanced frame of imaging information, the enhanced frame including data from each vector in the enhanced set of vectors.

Methods that include coherence filtering, envelope detection, and spatial filtering, can process raw high frequency image information received from the transducer into low frequency brightness data in which both high frequency and low frequency noise has been significantly reduced. Such methods can effectively reduce a full frequency range of image noise, with the frequency ranges affected by the high and low frequency noise reduction overlapping at least partially. Such noise reduction can significantly improve the SNR of the image information.

As noted, in many embodiments, the intravascular imaging system includes a display coupled to the imaging engine and configured to display an image generated by the image generator. Accordingly, in some embodiments, the spatially filtered brightness data can be displayed as an intravascular image on the display. As discussed herein, image information acquired at high frequencies tends to be less intense than that acquired at lower frequencies, and therefore may be difficult to observe in full detail on the display. However, because the processing steps prior to display have effectively reduced both the high and low frequency noise and significantly improved the SNR, gain can be applied to optionally amplify the spatially filtered brightness data for display without also amplifying noise so much so as to obscure the image (operation 618). Accordingly, such a system can take advantage of the high resolution obtainable by high frequency imaging while overcoming the drawbacks that are often associated with it.

In some systems, subsequent processing steps may be performed after spatially filtering the brightness data and prior to display. For example, gamma filtering may be employed to convert the data to the display ability of a monitor (if it is not already within the display ability) and determine an appropriate level of contrast to incorporate into the final image (operation 620).

In some embodiments, gamma filtering can help match the detection ability of the human eye, given that monitors can display a wider range of brightness/colors than the human eye can distinguish. Temporal Frame Filtering (TFF) can be employed, in which a series of images of substantially the same location are processed in order to smooth image regions showing relatively stable tissue features from frame to frame (e.g., vessel wall, lumen blood) and to maintain (not filter) information in image regions where tissue features vary from frame to frame (e.g., lumen border position changes) (operation 622). In many instances, TFF can perform local filtering rather than the same filtering across the entire frame.

Scan conversion can be employed in some example embodiments (operation 624). Scan conversion involves converting from polar coordinates to Cartesian coordinates. The data can be stored in a format that is independent of the anatomy of the vessel (e.g., data is independent of the rotation angle of the transducer). This data format can be called polar format (r-theta). The data can be mapped to the anatomy by scan conversion. Finally, an image may be generated based on the processed/filtered image information (operation 626).

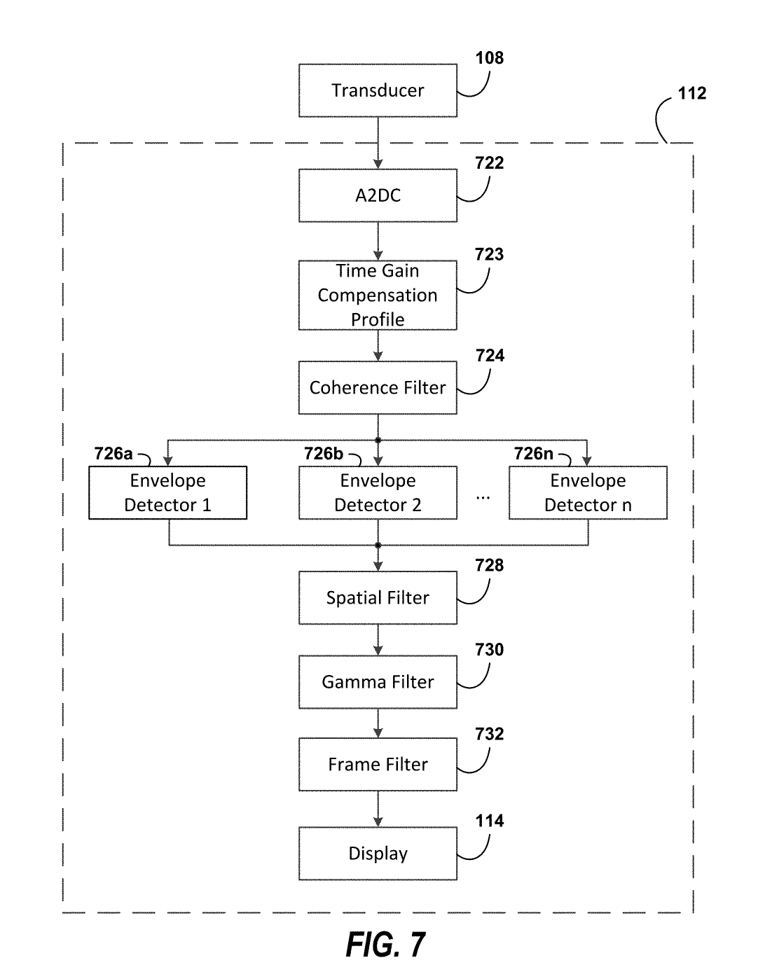

FIG. 7 is a data flow diagram illustrating the flow of image information from a transducer 108 to a display 114, according to an example embodiment. The transducer 108 can emit and receive energy within a patient's vasculature and can transmit high resolution and high frequency image information to the intravascular imaging engine 112 based on the received energy. In some embodiments, the signals comprise analog signals. In such embodiments, the analog signals can be passed through an analog-to-digital converter (A2DC) 722 to digitize the data into image information vectors. The resulting image information vectors can be in the form of a raw set of vectors that can be passed through a time gain compensation profile 723, which adjusts the data of the image information vectors based on whether the signal experienced attenuation by traveling through a blood-filled lumen or a blood-displaced lumen. The output of the time gain compensation profile 723 can then be passed through a coherence filter 724, which can reduce the amount of high frequency noise in the image information and produce a first set of vectors. In some instances, the first set of vectors can be designated as “super vectors,” and can include the same number of data points as associated vectors from the raw set of vectors. In some examples, the raw set of vectors consists of 4096 vectors, which are processed into 2048 “super vectors,” such that the first set of vectors consists of 2048 vectors.

“Super vectors” from the first set of vectors can be sent from the coherence filter 724 to a plurality of envelope detectors 726a, 726b, . . . 726n arranged in parallel to process the “super vectors” to generate a second set of vectors, which can be considered low frequency brightness vectors. In some examples, low frequency brightness vectors in the second set of vectors include fewer data points than the associated “super vectors” from the first set of vectors. In some examples, the reduction in the number of data points between the super vectors and the low frequency brightness vectors can be by a factor of five, ten, or other appropriate scaling factor so that each vector in the second set of vectors provides an accurate low-frequency representation of corresponding vectors in the first set of vectors. In some examples, the second set of vectors can include the same number of low frequency brightness vectors as the first set of vectors includes “super vectors.”

The brightness vectors in the second set of vectors may be passed to a spatial filter 728 configured to reduce the amount of low frequency noise in the brightness vectors. The spatial filter 728 can group the brightness vectors from the second set of vectors into processed vector groups. Data within the processed vector groups can be compared in the spatial filter 728 to generate an enhanced set of vectors, each vector including an enhanced set of data points. Spatially filtered brightness vectors in the enhanced set of vectors can include the same number of data points as the low frequency brightness vectors in the second set of vectors. In some examples, the spatial filter 728 can reduce the number of vectors. For instance, the spatial filter 728 can reduce the number of vectors in the second set of vectors by a factor of four in generating the enhanced set of vectors. That is, the enhanced set of vectors at the output of the spatial filter 728 can include one fourth as many vectors as the second set of vectors input into the spatial filter 728. The enhanced set of vectors can be combined to form an enhanced frame of imaging data.

The resulting spatially filtered brightness vectors in the enhanced set of vectors can be passed to an amplifier and/or subsequent processing components 730 such as gamma filtering or temporal frame filtering 732 to prepare the data for display. Components 730, 732 can include an image generator for generating an image based on the enhanced frame of imaging data. The generated image can be sent to display 114 for real-time display of a patient's vascular structure with reduced high frequency noise and reduced low frequency noise.

As described, exemplary intravascular imaging systems can include an intravascular imaging engine 112 configured to receive image information from a transducer 108 within a catheter assembly 102. The image information can be processed and displayed on a display 114. In many systems, the transducer 108 is configured to move longitudinally within the patient in order to image multiple locations. As such, it can be advantageous to generate a real-time display so that a user of the intravascular imaging system can observe the vascular structure as the transducer 108 moves through the patient. The processing steps as described herein can be performed in such a way so that high-resolution images generated from high frequency image information acquisition can be displayed in real time (or near real time) at a constant frame rate. In some embodiments, the frame rate can range from 30 to 60 frames per second. In some embodiments, the frame rate can be up to 160 frames per second.

In some intravascular imaging systems, a user can initiate any of the processing procedures described herein via the user interface 120. For example, a user can initiate high resolution, high frequency imaging via the user interface 120 and observe real-time high-resolution in vivo image information of a patient's vascular structure. The user can use this received information to translate the transducer 108 within the patient in a desired direction via the user interface 120 while continuing to observe the imaged structure. Processes herein described may be encoded in non-transitory computer-readable medium containing executable instructions for causing a processor to carry out such processes. Non-transitory computer-readable medium can be included in memory in the intravascular imaging engine 112.

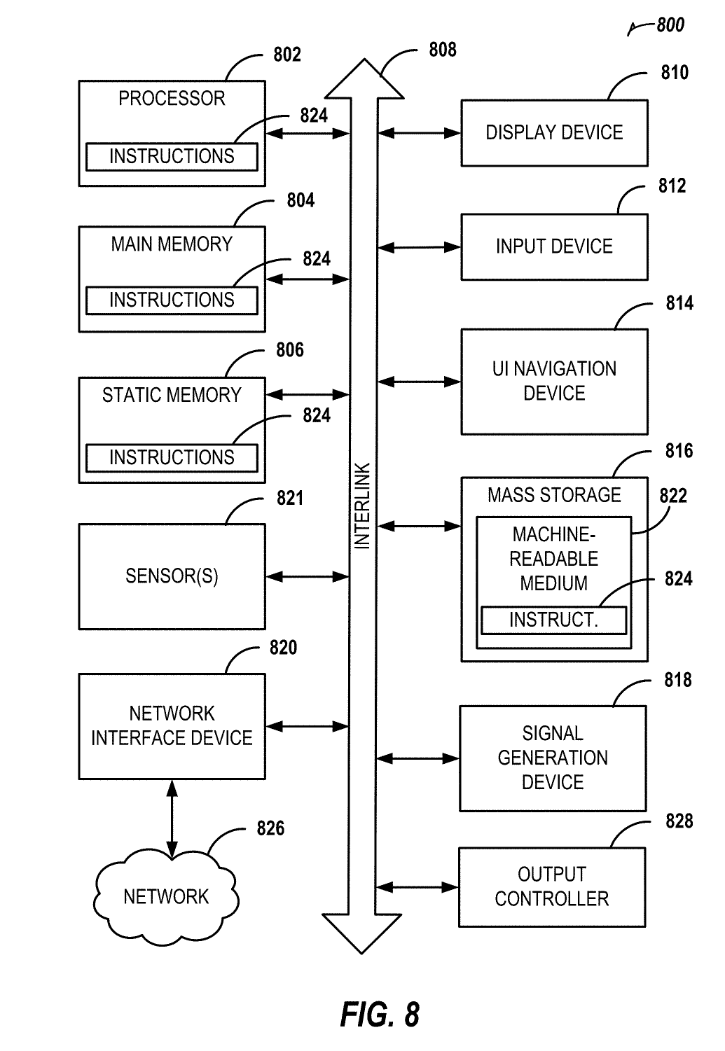

FIG. 8 is a block diagram illustrating an example of a machine 800, upon which any one or more example embodiments may be implemented. In alternative embodiments, the machine 800 may operate as a standalone device or may be connected (e.g., networked) to other machines. In a networked deployment, the machine 800 may operate in the capacity of a server machine, a client machine, or both in a client-server network environment. In an example, the machine 800 may act as a peer machine in a peer-to-peer (P2P) (or other distributed) network environment. The machine 800 may implement or include any portion of the systems, devices, or methods illustrated in FIGS. 1-7, and may be a computer, a server, or any machine capable of executing instructions (sequential or otherwise) that specify actions to be taken by that machine. Further, although only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations, etc.

Examples, as described herein, may include, or may operate by, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine-readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 800 may include a hardware processor 802 (e.g., a central processing unit (CPU), a graphics processing unit (GPU), a hardware processor core, or any combination thereof), a main memory 804 and a static memory 806, some or all of which may communicate with each other via an interlink (e.g., bus) 808. The machine 800 may further include a display unit 810, an alphanumeric input device 812 (e.g., a keyboard), and a user interface (UI) navigation device 814 (e.g., a mouse). In an example, the display unit 810, input device 812 and UI navigation device 814 may be a touch screen display. The machine 800 may additionally include a storage device (e.g., drive unit) 816, a signal generation device 818 (e.g., a speaker), a network interface device 820, and one or more sensors 821, such as a global positioning system (GPS) sensor, compass, accelerometer, or other sensor. The machine 800 may include an output controller 828, such as a serial (e.g., USB, parallel, or other wired or wireless (e.g., infrared (IR), near field communication (NFC), etc.) connection to communicate or control one or more peripheral devices (e.g., a printer, card reader, etc.) Machine 800 may be of one or more forms, such as a desktop computer, a laptop computer, a tablet computer, a smartphone, a smart watch, an all-in-one computer, a smart television, a digital table, etc.

The storage device 816 may include a machine-readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the storage device 816 may constitute machine-readable media.

Although the machine-readable medium 822 is illustrated as a single medium, the term “machine-readable medium” may include a single medium or multiple media (e.g., a centralized or distributed database, and/or associated caches and servers) configured to store the one or more instructions 824.

The term “machine-readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine-readable medium examples may include solid-state memories, and optical and magnetic media. Accordingly, machine-readable media are not transitory propagating signals. Specific examples of machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks.

The instructions 824 may further be transmitted or received over a communications network 826 using a transmission medium via the network interface device 820 utilizing any one of a number of transfer protocols (e.g., frame relay, Internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMAX®), IEEE 802.15.4 family of standards, Bluetooth®, Bluetooth® low energy technology, ZigBee®, peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Conventional terms in the fields of computer systems and computer networking have been used herein. The terms are known in the art and are provided only as a non-limiting example for convenience purposes. Accordingly, the interpretation of the corresponding terms in the claims, unless stated otherwise, is not limited to any particular definition.

Although specific embodiments have been illustrated and described herein, it will be appreciated by those of ordinary skill in the art that any arrangement that is calculated to achieve the same purpose may be substituted for the specific embodiments shown. Many adaptations will be apparent to those of ordinary skill in the art. Accordingly, this application is intended to cover any adaptations or variations.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. Moreover, in the following claims, the terms “first,” “second,” and “third,” etc. are used merely as labels, and are not intended to impose numerical requirements on their objects. In this document, a sensor set may include one or more sensors, which may be of different types. Furthermore, two different sensor sets may include one or more sensors that belong to both sensor sets.

In this Detailed Description, various features may have been grouped together to streamline the disclosure. This should not be interpreted as intending that an unclaimed disclosed feature is essential to any claim. Rather, inventive subject matter may lie in less than all features of a particular disclosed embodiment.

Various examples of systems and methods for intravascular imaging have been described. It will be appreciated that these and others are within the scope of the invention. If there is any conflict in the usages of a word or term in this specification and one or more patent(s) or other documents that may be incorporated herein by reference, the definitions that are consistent with this specification should be adopted.

The above description is intended to be illustrative, and not restrictive. For example, the above-described examples (or one or more aspects thereof) may be used in combination with each other. Other embodiments may be used, such as by one of ordinary skill in the art upon reviewing the above description.

What is claimed is:

1. A system comprising:
a catheter assembly including an intravascular imaging device, the intravascular imaging device including an imaging element to image a vasculature and generate imaging data; and
an imaging engine in communication with the intravascular imaging device, the imaging engine comprising a programmable processor, the imaging engine to:
determine a lumen state of the vasculature, the determined lumen state indicative of whether the vasculature is blood-filled or blood-cleared; and
generate an image based on the generated imaging data and the determined lumen state.

2. The system of claim 1, wherein the imaging engine further comprises a first time gain profile and a second time gain profile; and
wherein the imaging engine is to drive the intravascular imaging device with at least one of the first and second time gain profiles as a function of the determined lumen state.

3. The system of claim 1, wherein the imaging engine further comprises a first coherence filter and a second coherence filter; and
wherein the imaging engine is to generate the image by applying at least one of the first and second coherence filters as a function of the determined lumen state.

4. The system of claim 3, wherein the imaging engine is to execute envelope vector averaging with the first coherence filter when the determined lumen state is blood-filled.

5. The system of claim 3, wherein the imaging engine is to execute envelope vector averaging with the second coherence filter when the determined lumen state is blood-cleared.

6. The system of claim 1, wherein the imaging engine further comprises a first spatial filter and a second spatial filter; and
wherein the imaging engine is to generate the image by applying at least one of the first and second spatial filters as a function of the determined lumen state.

7. The system of claim 1, wherein the imaging engine further comprises a first gamma filter and a second gamma filter; and
wherein the imaging engine is to generate the image by applying at least one of the first and second gamma filters as a function of the determined lumen state.

8. The system of claim 1, wherein the imaging engine further comprises a first frame filter and a second frame filter; and
wherein the imaging engine is to generate the image by applying at least one of the first and second frame filters as a function of the determined lumen state.

9. A method comprising:
imaging a vasculature using an imaging element of an intravascular imaging device of a catheter assembly;
generating imaging data from a result of the imaging;
determining, using an imaging engine comprising a programmable processor, a lumen state of the vasculature, the determined lumen state indicative of whether the vasculature is blood-filled or blood-cleared; and
generating an image based on the generated imaging data and the determined lumen state.

10. The method of claim 9, wherein the imaging engine further comprises a first time gain profile and a second time gain profile, the method further comprising:
driving, using the imaging engine, the intravascular imaging device with at least one of a first and second time gain profiles as a function of the determined lumen state.

11. The method of claim 9, wherein the imaging engine further comprises a first coherence filter and a second coherence filter, the method further comprising:
generating, using the imaging engine, the image by applying at least one of the first and second coherence filters as a function of the determined lumen state.

12. The method of claim 9, wherein the imaging engine further comprises a first spatial filter and a second spatial filter, the method further comprising:
generating, using the imaging engine, the image by applying at least one of the first and second spatial filters as a function of the determined lumen state.

13. The method of claim 9, wherein the imaging engine further comprises a first gamma filter and a second gamma filter, the method further comprising:
generating, using the imaging engine, the image by applying at least one of the first and second gamma filters as a function of the determined lumen state.

14. The method of claim 9, wherein the imaging engine further comprises a first frame filter and a second frame filter, the method further comprising:
generating, using the imaging engine, the image by applying at least one of the first and second frame filters as a function of the determined lumen state.

15. A non-transitory computer-readable storage medium including instructions that, when executed by a computer, cause the computer to:
image a vasculature using an imaging element of an intravascular imaging device of a catheter assembly;
generate imaging data from a result of the imaging;
determine, using an imaging engine comprising a programmable processor, a lumen state of the vasculature, the determined lumen state indicative of whether the vasculature is blood-filled or blood-cleared; and
generate an image based on the generated imaging data and the determined lumen state.

16. The non-transitory computer-readable storage medium of claim 15, wherein the imaging engine further comprises a first time gain profile and a second time gain profile; and
wherein the instructions, when executed by the computer, further cause the computer to:
drive, using the imaging engine, the intravascular imaging device with at least one of a first and second time gain profiles as a function of the determined lumen state.

17. The non-transitory computer-readable storage medium of claim 15, wherein the imaging engine further comprises a first coherence filter and a second coherence filter; and
wherein the instructions, when executed by the computer, further cause the computer to:
generate, using the imaging engine, the image by applying at least one of the first and second coherence filters as a function of the determined lumen state.

18. The non-transitory computer-readable storage medium of claim 17, wherein the instructions, when executed by the computer, further cause the computer to:
execute, using the imaging engine, envelope vector averaging with the first coherence filter when the determined lumen state is blood-filled.

19. The non-transitory computer-readable storage medium of claim 17, wherein the instructions, when executed by the computer, further cause the computer to:
execute, using the imaging engine, envelope vector averaging with the second coherence filter when the determined lumen state is blood-cleared.

20. The non-transitory computer-readable storage medium of claim 15, wherein the imaging engine further comprises a first spatial filter and a second spatial filter; and
wherein the instructions, when executed by the computer, further cause the computer to:
generate, using the imaging engine, the image by applying at least one of the first and second spatial filters as a function of the determined lumen state.

21. The non-transitory computer-readable storage medium of claim 15, wherein the imaging engine further comprises a first gamma filter and a second gamma filter; and
wherein the instructions, when executed by the computer, further cause the computer to:
generate, using the imaging engine, the image by applying at least one of the first and second gamma filters as a function of the determined lumen state.

22. The non-transitory computer-readable storage medium of claim 15, wherein the imaging engine further comprises a first frame filter and a second frame filter; and
wherein the instructions, when executed by the computer, further cause the computer to:
generate, using the imaging engine, the image by applying at least one of the first and second frame filters as a function of the determined lumen state.
